(19) United States
(12) Patent Application Publication
(10) Pub. No.: US 2017/0000102 A1
(43) Pub. Date: Jan. 5, 2017

(54) Device for Evaporative Delivery of Volatile Substance

(71) Applicant: PPG Industries Ohio, Inc., Cleveland, OH (US)

(72) Inventor: Luciano M. Parrinello, Allison Park, PA (US)

(21) Appl. No.: 15/156,475

(22) Filed: May 17, 2016

Related U.S. Application Data
(60) Provisional application No. 62/163,069, filed on May 18, 2015.

(51) Int. Cl. A01M 1/20; C09D 129/04; C08J 7/04; A61L 9/12

(52) U.S. Cl. CPC A01M 1/2044; A61L 9/12; A01M 1/2055; C09D 129/04; C08J 7/047; A61L 2209/131; C08J 2300/22; C08J 2429/04

(57) ABSTRACT

The invention is directed to a device for evaporative delivery of volatile substances. The device includes (a) a reservoir portion containing a liquid volatile substance, the reservoir having an open cavity with a peripheral portion; (b) a microporous membrane having a first and second surface positioned over the reservoir, said membrane being affixed to the peripheral portion of the reservoir and wherein the second surface of the membrane contacts the liquid volatile substance, the microporous membrane further including a barrier coating layer over the first surface of the microporous membrane; and (c) a removable cap layer having a first surface and a second surface, wherein an adhesive layer is interposed between the first surface of the microporous membrane and the second surface of the cap layer such that the microporous vapor-permeable membrane and the liquid volatile substance are substantially sealed beneath the cap layer.

### CROSS REFERENCE TO RELATED APPLICATION

This application claims the benefit of priority of U.S. Provisional Application No. 62/163,069, filed May 18, 2015, which is incorporated by reference herein in its entirety.

### FIELD OF INVENTION

The present invention is directed to a device for the evaporative delivery of volatile substances, such as fragrances and insect repellants, to the immediate environment surrounding the same.

### BACKGROUND OF THE INVENTION

Membrane-based devices for the evaporative delivery of volatile substances, such as fragrances and insect repellants, into an ambient environment are known in the art. Such devices include four basic components: a volatile liquid reservoir, the volatile liquid, an evaporation membrane, and a peel-away (i.e., removable) cover layer. Prior to activation by removal of the cover layer, the liquid volatile material resides within the space created by the reservoir and the evaporation membrane. Generally, the evaporation surface of the evaporation membrane is completely covered by the cover layer and is sealed along the outer edge (or peripheral region) created by the edge of the reservoir and the evaporation membrane. The cover layer most often is provided with a pull tab to assist in removal of the cover layer, thereby activating the device.

A non-porous evaporation membrane typically is saturated by the liquid volatile substance prior to activation. Since this type of non-porous membrane is driven by gradient concentration, if the volatile liquid does not readily evaporate from the external side of the evaporation membrane, no further volatile liquid can be transported through the membrane. Further, a membrane driven by gradient concentration is dependent upon the amount of surface of the membrane in direct contact with the volatile liquid. Thus, once some of the volatile liquid is depleted from the reservoir, the maximum evaporation surface cannot be utilized. Once the level of the volatile liquid decreases with time, the evaporation rate decreases proportionally over the device life. Moreover, such non-porous membranes can be adversely affected by contact with many volatile substances. Thus, such non-porous membranes restrict the manufacturer of such devices to a limited number of volatile substance formulations.

Porous evaporation membranes have been used in such “peel and release” devices as well. The use of such porous membranes can overcome some of the deficiencies noted with the use of membranes driven by gradient concentrations. Porous membranes generally are driven by capillary action (as opposed to gradient concentration). These porous membranes allow for a broader range of volatile substance formulations. Further, such porous membranes provide the peel and release devices with a clear end of life indication. That is, all of the volatile liquid substance within the reservoir is depleted with a uniform delivery rate of vapor from the exterior surface of the porous membrane. Notwithstanding the aforementioned advantages, it has been found that the use of porous membranes in evaporative delivery devices can result in the collection of the liquid volatile material on the external surface of the membrane. In such instances, once the removable cover layer is peeled away, the membrane surface is wet and can drip onto the surrounding surfaces (e.g., furniture surfaces). This creates an unacceptable experience for the consumer. Therefore, there remains a need in the marketplace for devices for evaporative delivery of volatile substances which can overcome this problem.

### SUMMARY OF THE INVENTION

The present invention is directed to a device for evaporative delivery of volatile substances comprising:

(a) a reservoir portion containing a liquid volatile substance, the reservoir portion having an open cavity with a peripheral portion there around;

(b) a microporous, vapor-permeable membrane having a first surface and a second surface positioned over the reservoir, said membrane being affixed to the peripheral portion of the reservoir and wherein the second surface of the membrane is in contact with at least the liquid volatile substance, the microporous membrane comprising:
(A) a polymeric matrix,
(B) an interconnecting network of pores communicating throughout the polymeric matrix, and
(C) finely divided, substantially water-insoluble filler material,
wherein the microporous membrane further comprises a barrier coating layer over at least a portion of at least the first surface of the microporous membrane; and

(c) a removable cap layer having a first surface and a second surface, wherein an adhesive layer is interposed between the first surface of the microporous membrane and the second surface of the cap layer, such that the microporous vapor-permeable membrane and the liquid volatile substance are substantially sealed beneath the cap layer.

### DETAILED DESCRIPTION OF THE INVENTION

Unless otherwise indicated, all ranges disclosed herein are to be understood to encompass any and all subranges subsumed therein. For example, a stated range of “1 to 10” should be considered to include any and all subranges between (and inclusive of) the minimum value of 1 and the maximum value of 10; that is, all subranges beginning with a minimum value of 1 or more and ending with a maximum value of 10 or less, e.g., 1 to 6.1, 3.5 to 7.8, 5.5 to 10, etc.

Unless otherwise indicated, all numbers or expressions, such as those expressing structural dimensions, quantities of ingredients, etc., as used in the specification and claims are understood as modified in all instances by the term “about”.

As previously mentioned, the device of the present invention comprises a reservoir portion (a) containing a volatile substance. The term “volatile substance” as used herein and in the claims means a material that is capable of conversion to a gaseous or vapor form (i.e., capable of vaporizing) at ambient room temperature and pressure, in the absence of imparted additional or supplementary energy (e.g., in the form of heat and/or agitation). The volatile substance can comprise an organic volatile material, which can include those volatile materials comprising a solvent-based material, or those which are dispersed in a solvent-based material. The volatile substance typically is in a liquid form, but, in some instances, the volatile substance can be a solid form, and may be naturally occurring or synthetically formed. When in a solid form, the volatile substance typically sublimes from solid form to vapor form in the absence of an intermediate liquid form. The volatile substance may optionally be combined or formulated with non-volatile materials, such as a carrier (e.g., water and/or non-volatile solvents). In the case of a solid volatile substance, the non-volatile carrier may be in the form of a porous material (e.g., a porous inorganic material) in which the solid volatile material is held. Also, the solid volatile material may be in the form of a semi-solid gel. Typically, the volatile substance is in a liquid form.

The volatile substance can be, for example, a fragrance release material, such as a naturally occurring or synthetic perfume oil, an insect repellant release material, or mixtures thereof. For example, the volatile substance can be a fragrance release material in liquid form. Examples of perfume oils from which the volatile substance may be selected include, but are not limited to, oil of bergamot, bitter orange, lemon, mandarin, caraway, cedar leaf, clove leaf, cedar wood, geranium, lavender, orange, origanum, petitgrain, white cedar, patchouli, neroili, rose absolute, and combinations thereof. Examples of solid fragrance materials from which the volatile material may be selected include, but are not limited to, vanillin, ethyl vanillin, coumarin, tonalid, calone, heliotropene, musk xylol, cedrol, musk ketone benzophenone, raspberry ketone, methyl naphthyl ketone beta, phenyl ethyl salicylate, veltol, maltol, maple lactone, proeugenol acetate, evemyl, and combinations thereof.

The reservoir portion (a) comprises an open cavity with a peripheral portion there around. The reservoir portion can have any suitable shape and can be made from any suitable material. For example, the reservoir portion can comprise cellulosic materials, metal foils, polymeric materials or composites thereof. Naturally, the reservoir portion must be resistant to the volatile substance to be contained therein, i.e., it must not be made of a material which is chemically degraded, softened or swollen by the volatile substance. The reservoir portion should be suitably designed so as to define a cavity having a volume which can accommodate the desired amount of volatile substance and, if desired, a sufficient evaporation space. The cavity is open with an “edge” or peripheral portion there around the opening. One skilled in the art can envisage many variants of reservoir, both practical and decorative.

A microporous, vapor-permeable membrane (b), which has a first surface and a second surface, is positioned over the reservoir. The second surface of the membrane is in contact with at least the liquid volatile substance contained within the reservoir portion. For example, the microporous membrane (b) can be disposed over the reservoir open cavity and can extend to the peripheral portion there around the open cavity. The membrane can be affixed to the peripheral portion of the reservoir using any suitable adhesive material known in the art provided that the adhesive sufficiently penetrates the pores of the microporous membrane to prevent migration of the liquid volatile substance into the peripheral portion of the membrane. The membrane may be affixed to the peripheral portion of the reservoir using hot melt adhesives, such as those known in the art, or via known lamination techniques.

The microporous, vapor-permeable membrane (b) suitable for use in the device of the present invention generally comprises a polymeric matrix, an interconnecting network of pores communicating throughout the polymeric matrix, and finely divided, substantially water-insoluble filler material. The polymeric matrix of the membrane is composed of substantially water-insoluble thermoplastic organic polymer(s). The numbers and kinds of such polymers suitable for use as the matrix are large. In general, any substantially water-insoluble thermoplastic organic polymer which can be extruded, calendered, pressed, or rolled into film, sheet, strip, or web may be used. The polymer may be a single polymer or it may be a mixture of polymers. The polymers may be homopolymers, copolymers, random copolymers, block copolymers, graft copolymers, atactic polymers, isotactic polymers, syndiotactic polymers, linear polymers, or branched polymers. When mixtures of polymers are used, the mixture may be homogeneous or it may comprise two or more polymeric phases.

Examples of classes of suitable substantially water-insoluble thermoplastic organic polymers include thermoplastic polyolefins, poly(halo-substituted olefins), polyesters, polyamides, polyurethanes, polyureas, poly(vinyl halides), poly(vinylidene halides), polystyrenes, poly(vinyl esters), polycarbonates, polyethers, polysulfides, polyimides, polysilanes, polysiloxanes, polycaprolactones, polyacrylates, and polymethacrylates. Hybrid classes, from which the water-insoluble thermoplastic organic polymers may be selected include, for example, thermoplastic poly(urethane-ureas), poly(ester-amides), poly(silane-siloxanes), and poly(ether-esters) are within contemplation. Further examples of suitable substantially water-insoluble thermoplastic organic polymers include thermoplastic high density polyethylene, low density polyethylene, ultrahigh molecular weight polyethylene, polypropylene (atactic, isotactic, or syndiotactic), poly(vinyl chloride), polytetrafluoroethylene, copolymers of ethylene and acrylic acid, copolymers of ethylene and methacrylic acid, poly(vinylidene chloride), copolymers of vinylidene chloride and vinyl acetate, copolymers of vinylidene chloride and vinyl chloride, copolymers of ethylene and propylene, copolymers of ethylene and butene, poly(vinyl acetate), polystyrene, poly(omega-aminoundecanoic acid) poly(hexamethylene adipamide), poly(epsilon-caprolactam), and poly(methyl methacrylate). The recitation of these classes and example of substantially water-insoluble thermoplastic organic polymers is not exhaustive, and are provided for purposes of illustration.

Substantially water-insoluble thermoplastic organic polymers may in particular include, for example, poly(vinyl chloride), copolymers of vinyl chloride, or mixtures thereof. For example, the water-insoluble thermoplastic organic polymer can include an ultrahigh molecular weight polyolefin selected from ultrahigh molecular weight polyolefin (e.g., essentially linear ultrahigh molecular weight polyolefin) having an intrinsic viscosity of at least 10 deciliters/gram; or ultrahigh molecular weight polypropylene (e.g., essentially linear ultrahigh molecular weight polypropylene) having an intrinsic viscosity of at least 6 deciliters/gram; or a mixture thereof. In one example, the polymeric matrix comprises at least one polyolefin polymer. The water-insoluble thermoplastic organic polymer can include ultrahigh molecular weight polyethylene (e.g., linear ultrahigh molecular weight polyethylene) having an intrinsic viscosity of at least 18 deciliters/gram.

Ultrahigh molecular weight polyethylene (UHMWPE) is not a thermoset polymer having an infinite molecular weight, it is technically classified as a thermoplastic. However, because the molecules are substantially very long chains, UHMWPE softens when heated but does not flow as a molten liquid in a normal thermoplastic manner. The very long chains and the peculiar properties they provide to UHMWPE are believed to contribute in large measure to the desirable properties of microporous materials made using this polymer.

As indicated earlier, the intrinsic viscosity of the UHMWPE is at least about 10 deciliters/gram. Usually, the intrinsic viscosity is at least about 14 deciliters/gram. Often, the intrinsic viscosity is at least about 18 deciliters/gram. In many cases, the intrinsic viscosity is at least about 19 deciliters/gram. Although there is no particular restriction on the upper limit of the intrinsic viscosity, the intrinsic viscosity is frequently in the range of from about 10 to about 39 deciliters/gram. The intrinsic viscosity is often in the range of from about 14 to about 39 deciliters/gram. In most cases the intrinsic viscosity is in the range of from about 18 to about 39 deciliters/gram. An intrinsic viscosity in the range of from about 18 to about 32 deciliters/gram is preferred.

The nominal molecular weight of UHMWPE is empirically related to the intrinsic viscosity of the polymer according to the equation:

M(UHMWPE)=5.3×10⁴[η]^{1.37}

where M(UHMWPE) is the nominal molecular weight and [η] is the intrinsic viscosity of the UHMWPE expressed in deciliters/gram.

As used herein, intrinsic viscosity is determined by extrapolating to zero concentration the reduced viscosities or the inherent viscosities of several dilute solutions of the UHMWPE where the solvent is freshly distilled decahydronaphthalene to which 0.2 percent by weight, 3,5-di-tert-butyl-4-hydroxyhydrocinnamic acid, neopentanetetrayl ester [CAS Registry No. 6683-19-8] has been added. The reduced viscosities or the inherent viscosities of the UHMWPE are ascertained from relative viscosities obtained at 135° C. using an Ubbelohde No. 1 viscometer in accordance with the general procedures of ASTM D 4020-81, except that several dilute solutions of differing concentration are employed. ASTM D 4020-81 is, in its entirety, incorporated herein by reference.

In an exemplary embodiment, the matrix comprises a mixture of substantially linear ultrahigh molecular weight polyethylene having an intrinsic viscosity of at least 10 deciliters/gram, and lower molecular weight polyethylene having an ASTM D 1238-86 Condition E melt index of less than 50 grams/10 minutes and an ASTM D 1238-86 Condition F melt index of at least 0.1 gram/10 minutes. The nominal molecular weight of the lower molecular weight polyethylene (LMWPE) is lower than that of the UHMWPE. LMWPE is thermoplastic and many different types are known. One method of classification is by density, expressed in grams/cubic centimeter and rounded to the nearest thousandth, in accordance with ASTM D 1248-84 (re-approved 1989), as summarized as follows:

| Type | Abbreviation | Density (g/cm³) |
|---|---|---|
| Low Density Polyethylene | LDPE | 0.910-0.925 |
| Medium Density Polyethylene | MDPE | 0.926-0.940 |
| High Density Polyethylene | HDPE | 0.941-0.965 |

Any or all of these polyethylenes may be used as the LMWPE in the present invention. For some applications, HDPE may be used because it ordinarily tends to be more linear than MDPE or LDPE. ASTM D 1248-84 (Reapproved 1989) is, in its entirety, incorporated herein by reference.

Processes for making the various LMWPEs are well known and well documented. They include the high pressure process, the Phillips Petroleum Company process, the Standard Oil Company (Indiana) process, and the Ziegler process.

The ASTM D 1238-86 Condition E (that is, 190° C. and 2.16 kilogram load) melt index of the LMWPE is less than about 50 grams/10 minutes. Often, the Condition E melt index is less than about 25 grams/10 minutes. Preferably, the Condition E melt index is less than about 15 grams/10 minutes.

The ASTM D 1238-86 Condition F (that is, 190° C. and 21.6 kilogram load) melt index of the LMWPE is at least 0.1 gram/10 minutes. In many cases, the Condition F melt index is at least about 0.5 gram/10 minutes. Preferably, the Condition F melt index is at least about 1.0 gram/10 minutes.

ASTM D 1238-86 is, in its entirety, incorporated herein by reference.

Sufficient UHMWPE and LMWPE should be present in the matrix to provide their properties to the microporous material. One or more other thermoplastic organic polymers also may be present in the matrix so long as its presence does not materially affect the properties of the microporous material in an adverse manner. The other thermoplastic polymer may be one other thermoplastic polymer or it may be more than one other thermoplastic polymer. The amount of the other thermoplastic polymer which may be present depends upon the nature of such polymer. Examples of thermoplastic organic polymers which optionally may be present include poly(tetrafluoroethylene), polypropylene, copolymers of ethylene and propylene, copolymers of ethylene and acrylic acid, and copolymers of ethylene and methacrylic acid. If desired, all or a portion of the carboxyl groups of carboxyl-containing copolymers may be neutralized with sodium, zinc, or the like.

In most cases, the UHMWPE and the LMWPE together constitute at least about 65 percent by weight of the polymer of the matrix. Often the UHMWPE and the LMWPE together constitute at least about 85 percent by weight of the polymer of the matrix. Preferably, the other thermoplastic organic polymer is substantially absent so that the UHMWPE and the LMWPE together constitute substantially 100 percent by weight of the polymer of the matrix.

The UHMWPE can constitute at least one percent by weight of the polymer of the matrix, and the UHMWPE and the LMWPE together constitute substantially 100 percent by weight of the polymer of the matrix.

Where the UHMWPE and the LMWPE together constitute 100 percent by weight of the polymer of the matrix of the microporous material, the UHMWPE can constitute greater than or equal to 40 percent by weight of the polymer of the matrix. For example, the UHMWPE can constitute greater than or equal to 45 percent by weight of the polymer of the matrix. For example, the UHMWPE can constitute greater than or equal to 48 percent by weight of the polymer of the matrix. For example, the UHMWPE can constitute greater than or equal to 50 percent by weight of the polymer of the matrix. For example, the UHMWPE can constitute greater than or equal to 55 percent by weight of the polymer of the matrix. Also, the UHMWPE can constitute less than or equal to 99 percent by weight of the polymer of the matrix. For example, the UHMWPE can constitute less than or equal to 80 percent by weight of the polymer of the matrix. For example, the UHMWPE can constitute less than or equal to 70 percent by weight of the polymer of the matrix. For example, the UHMWPE can constitute less than or equal to 65 percent by weight of the polymer of the matrix. For example, the UHMWPE can constitute less than or equal to 60 percent by weight of the polymer of the matrix. The level of UHMWPE comprising the polymer of the matrix can range between any of these values inclusive of the recited values.

Likewise, where the UHMWPE and the LMWPE together constitute 100 percent by weight of the polymer of the matrix of the microporous material, the LMWPE can constitute greater than or equal to 1 percent by weight of the polymer of the matrix. For example, the LMWPE can constitute greater than or equal to 5 percent by weight of the polymer of the matrix. For example, the LMWPE can constitute greater than or equal to 10 percent by weight of the polymer of the matrix. For example, the LMWPE can constitute greater than or equal to 15 percent by weight of the polymer of the matrix. For example, the LMWPE can constitute greater than or equal to 20 percent by weight of the polymer of the matrix. For example, the LMWPE can constitute greater than or equal to 25 percent by weight of the polymer of the matrix. For example, the LMWPE can constitute greater than or equal to 30 percent by weight of the polymer of the matrix. For example, the LMWPE can constitute greater than or equal to 35 percent by weight of the polymer of the matrix. For example, the LMWPE can constitute greater than or equal to 40 percent by weight of the polymer of the matrix. For example, the LMWPE can constitute greater than or equal to 45 percent by weight of the polymer of the matrix. For example, the LMWPE can constitute greater than or equal to 50 percent by weight of the polymer of the matrix. For example, the LMWPE can constitute greater than or equal to 55 percent by weight of the polymer of the matrix. Also, the LMWPE can constitute less than or equal to 70 percent by weight of the polymer of the matrix. For example, the LMWPE can constitute less than or equal to 65 percent by weight of the polymer of the matrix. For example, the LMWPE can constitute less than or equal to 60 percent by weight of the polymer of the matrix. For example, the LMWPE can constitute less than or equal to 55 percent by weight of the polymer of the matrix. For example, the LMWPE can constitute less than or equal to 50 percent by weight of the polymer of the matrix. For example, the LMWPE can constitute less than or equal to 45 percent by weight of the polymer of the matrix. The level of the LMWPE can range between any of these values inclusive of the recited values.

It should be noted that for any of the previously described microporous materials of the present invention, the LMWPE can comprise high-density polyethylene.

The microporous material also includes a finely-divided, substantially water-insoluble particulate filler material. The particulate filler material may include an organic particulate material and/or an inorganic particulate material. The particulate filler material typically is not colored, for example, the particulate filler material is a white or off-white particulate filler material, such as a siliceous or clay particulate material.

The finely divided, substantially water-insoluble filler particles may constitute from 20 to 90 percent by weight of the microporous material. For example, such filler particles may constitute from 30 percent to 90 percent by weight of the microporous material. For example, such filler particles may constitute from 40 to 90 percent by weight of the microporous material. For example, such filler particles may constitute from 40 to 85 percent by weight of the microporous material. For example, such filler particles may constitute from 50 to 90 percent by weight of the microporous material. For example, such filler particles may constitute from 60 percent to 90 percent by weight of the microporous material.

The finely divided, substantially water-insoluble particulate filler may be in the form of ultimate particles, aggregates of ultimate particles, or a combination of both. At least about 90 percent by weight of the filler used in preparing the microporous material has gross particle sizes in the range of from 0.5 to about 200 micrometers, such as from 1 to 100 micrometers, as determined by the use of a laser diffraction particle size instrument, LS230 from Beckman Coulton, capable of measuring particle diameters as small as 0.04 micrometers. Typically, at least 90 percent by weight of the particulate filler has gross particle sizes in the range of from 10 to 30 micrometers. The sizes of the filler agglomerates may be reduced during processing of the ingredients used to prepare the microporous material. Accordingly, the distribution of gross particle sizes in the microporous material may be smaller than in the raw filler itself.

Non-limiting examples of suitable organic and inorganic particulate materials that may be used in the microporous material of the present invention include those described in U.S. Pat. No. 6,387,519 B1 at column 9, line 4 to column 13, line 62, the cited portions of which are incorporated herein by reference.

For example, the particulate filler material can comprise siliceous materials. Non-limiting examples of siliceous fillers that may be used to prepare the microporous material include silica, mica, montmorillonite, kaolinite, nanoclays such as cloisite available from Southern Clay Products, talc, diatomaceous earth, vermiculite, natural and synthetic zeolites, calcium silicate, aluminum silicate, sodium aluminum silicate, aluminum polysilicate, alumina silica gels, and glass particles. In addition to the siliceous fillers, other finely divided particulate substantially water-insoluble fillers optionally may also be employed. Non-limiting examples of such optional particulate fillers include carbon black, charcoal, graphite, titanium oxide, iron oxide, copper oxide, zinc oxide, antimony oxide, zirconia, magnesia, alumina, molybdenum disulfide, zinc sulfide, barium sulfate, strontium sulfate, calcium carbonate, and magnesium carbonate. For example, the siliceous filler may include silica and any of the aforementioned clays. Non-limiting examples of silicas include precipitated silica, silica gel, fumed silica, and combinations thereof.

Silica gel is generally produced commercially by acidifying an aqueous solution of a soluble metal silicate, e.g., sodium silicate at low pH with acid. The acid employed is generally a strong mineral acid such as sulfuric acid or hydrochloric acid, although carbon dioxide can be used. Inasmuch as there is essentially no difference in density between the gel phase and the surrounding liquid phase while the viscosity is low, the gel phase does not settle out, that is to say, it does not precipitate. Consequently, silica gel may be described as a non-precipitated, coherent, rigid, three-dimensional network of contiguous particles of colloidal amorphous silica. The state of subdivision ranges from large, solid masses to submicroscopic particles, and the degree of hydration from almost anhydrous silica to soft gelatinous masses containing on the order of 100 parts of water per part of silica by weight.

Precipitated silica generally is produced commercially by combining an aqueous solution of a soluble metal silicate, ordinarily alkali metal silicate such as sodium silicate, and an acid so that colloidal particles of silica will grow in a weakly alkaline solution and be coagulated by the alkali metal ions of the resulting soluble alkali metal salt. Various acids may be used, including but not limited to mineral acids. Non-limiting examples of acids that may be used include hydrochloric acid and sulfuric acid, but carbon dioxide can also be used to produce precipitated silica. In the absence of a coagulant, silica is not precipitated from solution at any pH. In a non-limiting embodiment, the coagulant used to effect precipitation of silica may be the soluble alkali metal salt produced during formation of the colloidal silica particles, or it may be an added electrolyte, such as a soluble inorganic or organic salt, or it may be a combination of both.

Precipitated silicas are available in many grades and forms from PPG Industries, Inc. These silicas are sold under the Hi-Sil® tradename.

For purposes of the present invention, the finely divided particulate substantially water-insoluble siliceous filler can comprise at least 50 percent by weight (e.g., at least 65 percent by weight, or at least 75 percent by weight), or at least 90 percent by weight of the substantially water-insoluble filler material. The siliceous filler may comprise from 50 to 90 percent by weight (e.g., from 60 to 80 percent by weight) of the particulate filler material, or the siliceous filler may comprise substantially all of the substantially water-insoluble particulate filler material.

The particulate filler (e.g., the siliceous filler) typically has a high-surface area, allowing the filler to carry much of the processing plasticizer composition used to produce the microporous material of the present invention. The filler particles are substantially water insoluble and also can be substantially insoluble in any organic processing liquid used to prepare the microporous material. This can facilitate retention of the particulate filler within the microporous material.

The microporous material of the present invention may also include minor amounts (e.g., less than or equal to 5 percent by weight, based on total weight of the microporous material) of other materials used in processing, such as lubricant, processing plasticizer, organic extraction liquid, water, and the like. Further materials introduced for particular purposes, such as thermal, ultraviolet and dimensional stability, may optionally be present in the microporous material in small amounts (e.g., less than or equal to 15 percent by weight, based on total weight of the microporous material). Examples of such further materials include, but are not limited to, antioxidants, ultraviolet light absorbers, reinforcing fibers such as chopped glass fiber strand, and the like. The balance of the microporous material, exclusive of filler and any coating, printing ink, or impregnant applied for one or more special purposes is essentially the thermoplastic organic polymer.

The microporous material of the present invention also includes a network of interconnecting pores, which communicate substantially throughout the microporous material. On a coating-free, printing ink-free and impregnant-free basis, pores typically constitute from 30 to 95 percent by volume, based on the total volume of the microporous material, when made by the processes as further described herein. The pores may constitute from 50 to 75 percent by volume of the microporous material, based on the total volume of the microporous material. As used herein and in the claims, the porosity (also known as void volume) of the microporous material, expressed as percent by volume, is determined according to the following equation:

Porosity=100[1−d₁/d₂]

where d₁ is the density of the sample, which is determined from the sample weight and the sample volume as ascertained from measurements of the sample dimensions; and d₂ is the density of the solid portion of the sample, which is determined from the sample weight and the volume of the solid portion of the sample. The volume of the solid portion of the microporous material is determined using a Quantachrome stereo pycnometer (Quantachrome Corp.) in accordance with the operating manual accompanying the instrument.

The volume average diameter of the pores of the microporous material is determined by mercury porosimetry using an Autoscan mercury porosimeter (Quantachrome Corp.) in accordance with the operating manual accompanying the instrument. The volume average pore radius for a single scan is automatically determined by the porosimeter. In operating the porosimeter, a scan is made in the high-pressure range (from 138 kilopascals absolute to 227 megapascals absolute). If 2 percent or less of the total intruded volume occurs at the low end (from 138 to 250 kilopascals absolute) of the high-pressure range, the volume average pore diameter is taken as twice the volume average pore radius determined by the porosimeter. Otherwise, an additional scan is made in the low pressure range (from 7 to 165 kilopascals absolute) and the volume average pore diameter is calculated according to the equation:

d=2[v₁r₁/w₁+v₂r₂/w₂]/[v₁/w₁+v₂/w₂]

where d is the volume average pore diameter; v₁ is the total volume of mercury intruded in the high pressure range; v₂ is the total volume of mercury intruded in the low pressure range; r₁ is the volume average pore radius determined from the high-pressure scan; r₂ is the volume average pore radius determined from the low-pressure scan; w₁ is the weight of the sample subjected to the high-pressure scan; and w₂ is the weight of the sample subjected to the low-pressure scan.

Generally, on a coating-free, printing ink-free and impregnant-free basis, the volume average diameter of the pores of the microporous material is at least 0.02 micrometers, typically at least 0.04 micrometers, and more typically at least 0.05 micrometers. On the same basis, the volume average diameter of the pores of the microporous material is also typically less than or equal to 0.5 micrometers, more typically less than or equal to 0.3 micrometers, and further typically less than or equal to 0.25 micrometers. The volume average diameter of the pores, on this basis, may range between any of these values, inclusive of the recited values. For example, the volume average diameter of the pores of the microporous material may range from 0.02 to 0.5 micrometers, or from 0.04 to 0.3 micrometers, or from 0.05 to 0.25 micrometers, in each case inclusive of the recited values.

In the course of determining the volume average pore diameter by means of the above-described procedure, the maximum pore radius detected may also be determined. This is taken from the low pressure range scan, if run; otherwise it is taken from the high pressure range scan. The maximum pore diameter of the microporous material is typically twice the maximum pore radius.

Coating, printing, and impregnation processes can result in filling at least some of the pores of the microporous material. In addition, such processes may also irreversibly compress the microporous material. Accordingly, the parameters with respect to porosity, volume average diameter of the pores, and maximum pore diameter are determined for the microporous material prior to application of one or more of these processes.

The microporous material can have a density of at least 0.7 g/cm³, or at least 0.8 g/cm³. As used herein, the density of the microporous material is determined by measuring the weight and volume of a sample of the microporous material. The upper limit of the density of the microporous material may range widely, provided it has an acceptable permeability to provide a sufficient evaporation rate for the volatile substance. Typically, the density of the microporous material is less than or equal to 1.5 g/cm³, or less than or equal to 1.0 g/cm³. The density of the microporous material can range between any of the above-stated values, inclusive of the recited values. For example, the microporous material can have a density of from 0.7 g/cm³ to 1.5 g/cm³, such as from 0.8 g/cm³ to 1.2 g/cm³, inclusive of the recited values.

Numerous art-recognized processes may be used to produce the microporous materials of the present invention. For example, the microporous material of the present invention can be prepared by mixing together filler particles, thermoplastic organic polymer powder, processing plasticizer and minor amounts of lubricant and antioxidant, until a substantially uniform mixture is obtained. The weight ratio of particulate filler to polymer powder employed in forming the mixture is essentially the same as that of the microporous material to be produced. The mixture, together with additional processing plasticizer, is typically introduced into the heated barrel of a screw extruder. Attached to the terminal end of the extruder is a sheeting die. A continuous sheet formed by the die is forwarded without drawing to a pair of heated calender rolls acting cooperatively to form a continuous sheet of lesser thickness than the continuous sheet exiting from the die. The level of processing plasticizer present in the continuous sheet at this point in the process can vary widely. For example, the level of processing plasticizer present in the continuous sheet, prior to extraction as described herein below, can be greater than or equal to 30 percent by weight of the continuous sheet, such as greater than or equal to 40 percent by weight, or greater than or equal to 45 percent by weight of the continuous sheet prior to extraction. Also, the amount of processing plasticizer present in the continuous sheet prior to extraction can be less than or equal to 70 percent by weight of the continuous sheet, such as less than or equal to 65 percent by weight, or less than or equal to 60 percent by weight, or less than or equal to 55 percent by weight of the continuous sheet prior to extraction. The level of processing plasticizer present in the continuous sheet at this point in the process, prior to extraction, can range between any of these values inclusive of the recited values.

The continuous sheet from the calender is then passed to a first extraction zone where the processing plasticizer is substantially removed by extraction with an organic liquid, which is a good solvent for the processing plasticizer, a poor solvent for the organic polymer, and more volatile than the processing plasticizer. Usually, but not necessarily, both the processing plasticizer and the organic extraction liquid are substantially immiscible with water. The continuous sheet then passes to a second extraction zone where residual organic extraction liquid is substantially removed by steam and/or water. The continuous sheet is then passed through a forced air dryer for substantial removal of residual water and remaining residual organic extraction liquid. From the dryer, the continuous sheet, which is microporous material, is passed to a take-up roll.

The processing plasticizer is a liquid at room temperature and usually is a processing oil, such as paraffinic oil, naphthenic oil, or aromatic oil. Suitable processing oils include those meeting the requirements of ASTM D 2226-82, Types 103 and 104. More typically, processing oils having a pour point of less than 220° C. according to ASTM D 97-66 (re-approved 1978), are used to produce the microporous material of the present invention. Processing plasticizers useful in preparing the microporous material of the present invention are discussed in further detail in U.S. Pat. No. 5,326,391 at column 10, lines 26 through 50, which disclosure is incorporated herein by reference.

The processing plasticizer composition used to prepare the microporous material can have little solvating effect on the polyolefin at 60° C., and only a moderate solvating effect at elevated temperatures on the order of 100° C. The processing plasticizer composition generally is a liquid at room temperature. Non-limiting examples of processing oils that may be used can include SHELLFLEX® 412 oil, SHELLFLEX® 371 oil (Shell Oil Co.), which are solvent refined and hydrotreated oils derived from naphthenic crude oils, ARCOprime® 400 oil (Atlantic Richfield Co.) and KAYDOL® oil (Witco Corp.), which are white mineral oils. Other non-limiting examples of processing plasticizers can include phthalate ester plasticizers, such as dibutyl phthalate, bis(2-ethylhexyl) phthalate, diisodecyl phthalate, dicyclohexyl phthalate, butyl benzyl phthalate, and ditridecyl phthalate. Mixtures of any of the foregoing processing plasticizers can be used to prepare the microporous material of the present invention.

There are many organic extraction liquids that can be used to prepare the microporous material of the present invention. Examples of other suitable organic extraction liquids include those described in U.S. Pat. No. 5,326,391 at column 10, lines 51 through 57, which disclosure is incorporated herein by reference.

The extraction fluid composition can comprise halogenated hydrocarbons, such as chlorinated hydrocarbons and/or fluorinated hydrocarbons. In particular, the extraction fluid composition may include halogenated hydrocarbon(s) and have a calculated solubility parameter coulomb term (δclb) ranging from 4 to 9 (Jcm³)^{1/2}. Non-limiting examples of halogenated hydrocarbon(s) suitable as the extraction fluid composition for use in producing the microporous material of the present invention can include one or more azeotropes of halogenated hydrocarbons selected from trans-1,2-dichloroethylene, 1,1,1,2,2,3,4,5,5,5-decafluoropentane, and/or 1,1,1,3,3-pentafluorobutane. Such materials are available commercially as VERTREL MCA (a binary azeotrope of 1,1,1,2,2,3,4,5,5,5-dihydrodecafluoropentane and trans-1,2-dichloroethylene: 62%/38%) and VERTREL CCA (a ternary azeotrope of 1,1,1,2,2,3,4,5,5,5-dihydrodecafluoropentane, 1,1,1,3,3-pentafluorobutane, and trans-1,2-dichloroethylene: 33%/28%/39%), both available from MicroCare Corporation.

The residual processing plasticizer content of microporous material according to the present invention is usually less than 10 percent by weight, based on the total weight of the microporous material, and this amount may be further reduced by additional extractions using the same or a different organic extraction liquid. Often, the residual processing plasticizer content is less than 5 percent by weight, based on the total weight of the microporous material, and this amount may be further reduced by additional extractions.

The microporous material of the present invention may also be produced according to the general principles and procedures of U.S. Pat. Nos. 2,772,322; 3,696,061; and/or 3,862,030. These principles and procedures are particularly applicable where the polymer of the matrix is or is predominately poly(vinyl chloride) or a copolymer containing a large proportion of polymerized vinyl chloride.

Microporous materials produced by the above-described processes optionally may be stretched. Stretching of the microporous material typically results in both an increase in the void volume of the material, and the formation of regions of increased or enhanced molecular orientation. As is known in the art, many of the physical properties of molecularly oriented thermoplastic organic polymer, including tensile strength, tensile modulus, Young's modulus, and others, differ (e.g., considerably) from those of the corresponding thermoplastic organic polymer having little or no molecular orientation. Stretching is typically accomplished after substantial removal of the processing plasticizer as described above.

Various types of stretching apparatus and processes are well known to those of ordinary skill in the art, and may be used to accomplish stretching of the microporous material of the present invention. Stretching of the microporous materials is described in further detail in U.S. Pat. No. 5,326,391 at column 11, line 45 through column 13, line 13, which disclosure is incorporated herein by reference.

The microporous membrane further comprises at least one barrier coating layer over at least one of the first and second surfaces of the microporous membrane. In a particular embodiment of the present invention, the microporous membrane comprises a barrier coating layer over at least the first surface of the microporous membrane.

The barrier coating layer(s) can be formed from a coating composition selected from liquid coating compositions and solid particulate coating compositions (e.g., powder coating compositions). Typically, the barrier coating layer(s) are formed from a liquid coating composition which may optionally include a solvent selected from water, organic solvents, and combinations thereof. The barrier coating layer(s) may be selected from crosslinkable coating compositions (e.g., thermosetting coating compositions and photo-curable coating compositions), and non-crosslinkable coating compositions (e.g., air-dry coating compositions). The barrier coating layer(s) may be applied to the respective surfaces of the microporous material in accordance with art-recognized methods, such as spray application, curtain coating, dip coating, and/or drawn-down coating (e.g., by means of a doctor blade or draw-down bar) techniques.

The coating compositions each independently can include art-recognized additives, such as antioxidants, ultraviolet light stabilizers, flow control agents, dispersion stabilizers (e.g., in the case of aqueous dispersions), and colorants (e.g., dyes and/or pigments). Typically, the barrier coating compositions are free of colorants and, as such, are substantially clear or opaque. Optional additives may be present in the coating compositions in individual amounts of from, for example, 0.01 to 10 percent by weight, based on the total weight of the barrier coating composition.

The barrier coating layer(s) can be formed from an aqueous coating composition that includes dispersed organic polymeric material. The aqueous coating composition may have a particle size of from 200 to 400 nm. The solids of the aqueous coating composition may vary widely, for example from 0.1 to 30 percent by weight, or from 1 to 20 percent by weight, in each case based on total weight of the aqueous coating composition. The organic polymers comprising the aqueous coating composition may have number average molecular weights (Mn) of, for example, from 1000 to 4,000,000, or from 10,000 to 2,000,000.

The aqueous coating composition can be selected, for example, from aqueous poly(meth)acrylate dispersions, aqueous polyurethane dispersions, aqueous silicone (or silicon) oil dispersions, and combinations thereof. The poly(meth)acrylate polymers of the aqueous poly(meth)acrylate dispersions may be prepared in accordance with art-recognized methods. For example, the poly(meth)acrylate polymers may include residues (or monomer units) of alkyl (meth)acrylates having from 1 to 20 carbon atoms in the alkyl group. Examples of alkyl (meth)acrylates having from 1 to 20 carbon atoms in the alkyl group include, but are not limited to, methyl (meth)acrylate, ethyl (meth)acrylate, 2-hydroxyethyl (meth)acrylate, propyl (meth)acrylate, 2-hydroxypropyl (meth)acrylate, isopropyl (meth)acrylate, butyl (meth)acrylate, isobutyl (meth)acrylate, tert-butyl (meth)acrylate, 2-ethylhexyl (meth)acrylate, lauryl (meth)acrylate, isobornyl (meth)acrylate, cyclohexyl (meth)acrylate, and 3,3,5-trimethylcyclohexyl (meth)acrylate. For purposes of non-limiting illustration, an example of an aqueous poly(meth)acrylate dispersion from which the coating composition may be selected is HYCAR 26138, which is commercially available from Lubrizol Advanced Materials, Inc.

The polyurethane polymers of the aqueous polyurethane dispersions, from which the barrier coating layer(s) may be selected, include any of those known to the skilled artisan. Typically, the polyurethane polymers are prepared from isocyanate functional materials having two or more isocyanate groups, and active hydrogen functional materials having two or more active hydrogen groups. The active hydrogen groups may be selected from, for example, hydroxyl groups, thiol groups, primary amines, secondary amines, and combinations thereof. For purposes of non-limiting illustration, a suitable example of an aqueous polyurethane dispersion is WITCOBOND W-240, which is commercially available from Chemtura Corporation.

The silicon polymers of the aqueous silicone oil dispersions may be selected from known and art-recognized aqueous silicone oil dispersions. For purposes of non-limiting illustration, an example of an aqueous silicon dispersion from which the barrier coating composition may be independently selected is MOMENTIVE LE-410, which is commercially available from Momentive Performance Materials.

In a further embodiment of the present invention, the barrier coating layer can be formed from a coating composition comprising a resinous component selected from the group consisting of polyvinyl alcohols, polyvinyl ethers, polyurethanes, polyureas, polyamides, polyvinylidene chlorides, epoxy-amine polymers, poly(meth)acrylates, polyesters, and mixtures thereof. In a particular example, the barrier coating layer(s) are formed from a coating composition comprising poly(vinyl alcohol).

The poly(vinyl alcohol)-containing barrier coating may be formed from liquid coating compositions which may optionally include a solvent selected from water, organic solvents, and combinations thereof. The poly(vinyl alcohol) coating may be selected from crosslinkable coatings (e.g., thermosetting coatings), and non-crosslinkable coatings (e.g., air-dry coatings). The poly(vinyl alcohol) coating may be applied to the respective surfaces of the microporous material in accordance with art-recognized methods, such as spray application, curtain coating, or drawn-down coating (e.g., by means of a doctor blade or draw-down bar).

In an example, the poly(vinyl alcohol) coatings are each independently formed from aqueous poly(vinyl alcohol) coating compositions. The solids of the aqueous poly(vinyl alcohol) coating composition may vary widely, for example from 0.1 to 15 percent by weight, or from 0.5 to 9 percent by weight, in each case based on total weight of the aqueous coating composition. The poly(vinyl alcohol) polymer present in the poly(vinyl alcohol) coating compositions may have number average molecular weights (Mn) of, for example, from 100 to 1,000,000, or from 1,000 to 750,000.

The poly(vinyl alcohol) polymer may be a homopolymer or copolymer. Co-monomers from which the poly(vinyl alcohol) copolymer may be prepared include those which are co-polymerizable (by means of radical polymerization) with vinyl acetate, and which are known to the skilled artisan. For purposes of illustration, co-monomers from which the poly(vinyl alcohol) copolymer may be prepared include, but are not limited to: (meth)acrylic acid, maleic acid, fumaric acid, crotonic acid, metal salts thereof, alkyl esters thereof (e.g., C₂-C₁₀ alkyl esters thereof), polyethylene glycol esters thereof, and polypropylene glycol esters thereof; vinyl chloride; tetrafluoroethylene; 2-acrylamido-2-methyl-propane sulfonic acid and its salts; acrylamide; N-alkyl acrylamide; N,N-dialkyl substituted acrylamides; and N-vinyl formamide.

A non-limiting example of a suitable poly(vinyl alcohol) coating composition that may be used to form the poly(vinyl alcohol) coated microporous material of the present invention is SELVOL® 325, which is commercially available from Sekisui Specialty Chemicals.

Any of the aforementioned coating compositions used to form the barrier coating layer(s) may each independently include art-recognized additives, such as antioxidants, ultraviolet light stabilizers, flow control agents, dispersion stabilizers (e.g., in the case of aqueous dispersions), plasticizers, and the like. Optional additives may be present in the poly(vinyl alcohol) coating compositions in individual amounts of from, for example, 0.01 to 10 percent by weight, based on the total weight of the coating composition.

Suitable compositions for forming the barrier coating layer(s) used in the device of the present invention also can include those described in U.S. Patent Application Publication No. 2005/0196601 A1 at paragraphs [0011]-[0036], the cited portions of which being incorporated by reference herein.

Any of the previously mentioned barrier coating layer(s) each independently can be applied at any suitable thickness, provided the microporous material has a vapor permeability sufficient to provide a consistent and uniform vapor delivery rate. Also, the barrier coating layer is present on at least the first surface of the microporous membrane at a coating weight (i.e., weight of the coating which has been applied to a surface of the microporous material) of from 0.5 to 5.50 g/m², such as from 0.75 to 5 g/m², or from 1.0 to 3 g/m² .

It should be noted that the barrier coating layer, if desired, can further comprise a high-aspect ratio pigment selected from the group consisting of vermiculite, mica, talc, metal flakes, platy clays, and platy silicas. The high-aspect ratio pigments or platelets can be present in the compositions used to form the barrier coating layer(s) in amounts from 0.1 to 20 weight percent of the composition, such as from 1 to 10 weight percent, with weight percent based on the total solid weight of the coating composition. The high-aspect ratio pigments/platelets may form a “fish-scale” arrangement within the coating layer which provides a tortuous path for the vapor to pass through from one side of the coating layer to the other. Such pigments/platelets typically have diameters ranging from 0 to 20 microns, such as from 2 to 5 microns, or from 2 to 10 microns. The aspect ratio of the pigment/platelets typically is at least 5:1, such as at least 10:1, or 20:1. The amount of high-aspect ratio pigment/platelets will be determined based on the desired properties of barrier and/or flexibility/elasticity to be achieved with the coating.

The barrier coating composition can form the barrier coating layer(s) at ambient temperature or elevated temperature, depending upon the coating composition components.

The device for evaporative delivery of volatile substances of the present invention further comprises a removable cap layer (c) having a first surface and a second surface. An adhesive layer is interposed between the first surface of the microporous membrane and the second surface of the cap layer such that the microporous vapor-permeable membrane and the liquid volatile substance are substantially sealed beneath the cap layer.

For example, the adhesive layer can be applied to the second surface of the cap layer (c) and the cap layer then affixed to the reservoir portion. The adhesive layer can be applied to the second surface of the cap layer in such a way that the adhesive layer is in contact with the peripheral portion of the vapor-permeable membrane. In another example, the adhesive layer can be applied to the entire second surface of the cap layer such that, when affixed to the vapor-permeable membrane, the adhesive layer is in contact with the first surface of the microporous, vapor-permeable membrane which includes the barrier coating layer. In another example, the adhesive layer can be applied to the peripheral portion of the first surface of the membrane or to the entire first surface of the membrane, to which the second surface of the cap layer is adhered.

The removable cap layer (c) can be a peel seal which, optionally, comprises a tab pull in order to facilitate removal from the device, thereby exposing the microporous, vapor-permeable membrane to activate the evaporative delivery of the volatile substance. The cap layer (c) can comprise metal foils, polymeric films, carbon films, silver/carbon films, coated paper, and the like. Typically, the removable cap layer (c) comprises at least one layer selected from the group consisting of metal foils, polymeric films, and combinations thereof. For example, the cap layer can comprise at least one polymeric film which has been printed or coated to appear metallized or “foil-like”. Any know metal foils can be used, provided desired properties are achieved. Suitable polymeric films can include, but are not limited to, polyethylene film, polypropylene film, poly(ethylene terephthalate) film, polyester film, polyurethane film, poly(ester/urethane) film, or poly(vinyl alcohol) films. Any suitable polymeric film can be used, provided the desired properties are achieved. The cap layer (c) also can comprise a metallized polymeric film either alone or in combination with a metal foil layer, a polymeric film layer, or both. The cap layer can comprise one layer or more than one layer in any combination.

The adhesive layer can comprise any of the known adhesives provided that the adhesive provides sufficient tack to keep the device sealed until activation by the consumer, while maintaining the removability of the cap layer. In a particular embodiment, the adhesive layer comprises a pressure-sensitive adhesive (“PSA”), such as any of the PSA materials known in the art. Suitable PSA materials include rubber-based adhesives, block co-polymer adhesives, polyisobutene-based adhesives, acrylic-based adhesives, silicone-based adhesives, polyurethane-based adhesives, vinyl-based adhesives, and mixtures thereof.

The present invention is more particularly described in the examples that follow, which are intended to be illustrative only, since numerous modifications and variations therein will be apparent to those skilled in the art. Unless otherwise specified, all parts and percentages are by weight.

### EXAMPLES

### Part 1. Barrier Coating Preparation

A barrier coating solution was prepared by dispersing 40 g SELVOL® 325 (a hydrolyzed poly(vinyl alcohol) available from Sekisui Specialty Chemicals) in 667 g cool water under mild agitation in a 1000 mL beaker. The mixture was heated to 190° F. (87.8° C.) and stirred for 20-30 minutes until completely dissolved. The resultant solution was allowed to cool to room temperature while stirring, yielding a homogeneous solution with 6% measured solids by weight. This solution was diluted further to prepare coating solutions used in Part 2.

### Part 2. Preparation of Coated Microporous Membrane Sheets

Sheets of TESLIN® SP (10 mil (0.25 mm) thickness, “SP”) or TESLIN HD (11 mil (0.28 mm) thickness, “HD”), both available from PPG Industries, Inc., were first weighed, then placed on a clean glass surface. The top corners were taped to the glass, and a piece of clear 10 mil thick polyester 11″×3″ (27.94 cm×7.62 cm) was taped to the glass surface, positioned to cover the top ½″ (1.3 cm) of the sheet. For each of the sheets coated, the barrier coating solution from Part 1 was diluted to the indicated solids. A ½-inch (1.3 cm) wire wrapped metering rod, of the types specified in Table 1 from Diversified Enterprises, was placed parallel to the top edge, near the top edge of the polyester. A 10-20 mL quantity of coating was deposited as a bead strip (approximately ¼ inch (0.6 cm) wide) directly next to and touching the metering rod using a disposable pipette. The bar was drawn completely across the sheet, attempting a continuous/constant rate, applying the composition to the entire exposed surface of the sheet. The resultant wet sheet was removed from the glass surface, immediately weighed, the wet coating weight recorded, then the coated sheet was placed in a forced air oven and dried at 95° C. for 2 minutes. The dried sheet was removed from the oven and the coating procedure was repeated onto the same coated sheet surface. The two wet coating weights were used to calculate the final dry coating weight in grams per square meter. The coated sheets are described in Table 1.

The following formula was used to calculate the final dry coating weight:

Coating Weight (g/m²)=((coatings solids×0.01)×(1st wet coating wt. (g)+2nd wet coating wt. (g)))/(surface area coated (m²))

TABLE 1
Microporous sheets for testing

| | TESLIN | Substrate | | | Wire | Calculated Final |
|---|---|---|---|---|---|---|
| | Substrate | thickness | | Coating | Wrapped | Coating Weight |
| Example # | Type | (mil) | Sheet Size | solids | Rod # | (g/m²) |
| 1 | SP | 10 | A4 | 2.0 | 2.5 | 0.6 |
| 2 | SP | 10 | A4 | 5.9 | 9 | 2.0 |
| 3 | HD | 11 | 8.5″ × 11″ | 3.7 | 3 | 1.1 |
| 4 | HD | 11 | 8.5″ × 11″ | 5.0 | 3 | 1.5 |
| 5 | HD | 11 | 8.5″ × 11″ | 4.5 | 12 | 2.4 |
| 6 | SP | 10 | 8.5″ × 11″ | 4.1 | 3 | 1.0 |

| | | | | |
|---|---|---|---|---|
| CE-7 | SP | 10 | 8.5″ × 11″ | No coating |
| CE-8 | HD | 11 | 8.5″ × 11″ | No coating |

### Part 3. Assembly of Simulated Peel and Release Device

The holder assembly used for evaporation rate and performance testing of a membrane consisted of a front clamp with a ring gasket, a back clamp, test reservoir cup, and four screws. The test reservoir cup was fabricated from a clear thermoplastic polymer having interior dimensions defined by a circular diameter at the edge of the open face of approximately 4 centimeters and a depth of no greater than 1 centimeter. The open face was used to determine the volatile material transfer rate. Each clamp of the holder assembly had a 1.5″ (3.8 cm) diameter circular opening to accommodate the test reservoir cup and provide an opening to expose the membrane under test. When placing a membrane under test, i.e., a sheet of microporous material having a thickness of from 6 to 18 mils (0.15 to 0.46 mm), the back clamp of the holder assembly was placed on top of a cork ring. The test reservoir cup was placed in the back clamp and charged with approximately 2 mL of benzyl acetate. An approximately 2″ (5.1 cm) diameter disk was cut out of the membrane sheet. A 2″×2″ (5 cm×5 cm) square of label material (specified in the Tables following) was applied to the membrane disc. When a coated microporous sheet was to be tested, the label material was applied to the coated surface. The membrane/label assembly was placed directly over and in contact with the edge of the reservoir cup such that 12.5 cm² of the volatile material contact surface of the microporous sheet was exposed to the interior of the reservoir and the label side was exposed to the atmosphere. The front clamp of the holder was carefully placed over the entire assembly. The screws were attached and tightened enough such that the gasket formed a leak-free seal. The holder was labeled to identify the membrane sample under test. From 3 to 5 replicates were prepared for each test, including control (uncoated) samples. All samples within a group were tested at the same time to minimize noise from differences in atmospheric conditions.

### Part 4. Testing of Simulated Devices

The testing was performed in three steps: conditioning, activation/equilibration, and evaporation rate measurement. To condition the assemblies, each group of devices were positioned such that the membrane surfaces were vertical (i.e., the liquid was in contact with at least a portion of the membrane). The amount of time (in days) each group was held in this position is recorded in the following tables as “conditioning time”. Following the given conditioning time, all of the assemblies within a group were placed horizontal and the clamps removed. To activate, each label was carefully peeled away, noting the appearance of the membrane surface underneath the label. The appearance was rated as follows: 1—wet with accumulated liquid; 2—uniformly wet; 3—areas of wet and dry; 4—uniformly dry. The devices were then reassembled without the labels. Each holder assembly was weighed to obtain an initial weight of the entire charged assembly. The assemblies were then placed, standing upright, in a laboratory chemical fume hood having approximate dimensions of 5 feet (1.5 m) in height×5 feet (1.5 m) in width×2 feet (0.6 m) in depth. With the test reservoir standing upright, benzyl acetate was in direct contact with at least a portion of the volatile material contact surface of the microporous sheet. The glass doors of the fume hood were pulled down, and the air flow through the hood was adjusted to have approximately eight (8) turns of hood volume per hour. The temperature in the hood was maintained at 25° C.±5° C., with ambient humidity. The test reservoirs were regularly weighed in the hood. Immediately after activation, the devices were allowed an equilibration time of three to five days before determining the steady state evaporation rate.

The calculated weight loss of benzyl acetate, in combination with the elapsed time and surface area of the microporous sheet exposed to the interior of the test reservoir, were used to determine the volatile material transfer rate of the microporous sheet, in units of mg/(hour*cm²). The average evaporation rate (mg/hr) of the replicates was reported for the entire assembly in the Tables below. These two values are related by the following formula:

Average evaporation rate (mg/hr)/12.5 cm²=volatile material transfer rate (mg/(hour*cm²))

Tables 2 and 3 list the results for the two substrate types at various coating weights versus an uncoated control. Table 4 shows results for three different pressure-sensitive adhesives on the same substrate with the same coating weight. In all cases, the labels were removed without difficulty.

TABLE 2
10 mil substrate; 3 day conditioning time

| | Label | Membrane | Delivery Rate¹ |
|---|---|---|---|
| Sample # | type | Appearance | (mg/hr) |
| CE-7 | M-713² | 1 | 4.8 |
| 1 | M-713 | 1 | 4.2 |
| 2 | M-713 | 4 | 3.9 |

¹5 days equilibration prior to measuring delivery rate.
²A metallic label with a clear solvent acrylic, available from General Data Company, Inc.

TABLE 3
11 mil substrate; 6 day conditioning time

| | Label | Membrane | Free | Delivery Rate¹ |
|---|---|---|---|---|
| Sample # | Type | Appearance | Liquid | (mg/hr) |
| CE-8 | M-713 | 1 | Yes | 4.6 |
| 3 | M-713 | 2 | No | 3.2 |
| 4 | M-713 | 2 | No | 2.0 |
| 5 | M-713 | 4 | No | 1.6 |

¹5 days equilibration prior to measuring delivery rate.

TABLE 4
Pressure-Sensitive Adhesive Labels

| | | Conditioning Time | | | |
|---|---|---|---|---|---|
| | | 7 Days | | 28 Days | |
| | | | Delivery | | Delivery |
| | Label | | Rate¹ | | Rate¹ |
| Sample # | Type | Appearance | (mg/hr) | Appearance | (mg/hr) |
| 6 | M-713 | 3 | 0.9 | 3 | 0.9 |
| 6 | A31² | 3 | 0.7 | 3 | 1.1 |
| 6 | SSX³ | 3 | 0.7 | 3 | 1.2 |

¹3 days equilibration prior to measuring delivery rate.
²A polyester label with an aggressive solvent acrylic adhesive, available from General Data Company, Inc.
³A polyester label with a silinated polyurethane adhesive, available from General Data Company, Inc.

Whereas particular embodiments of this invention have been described above for purposes of illustration, it will be evident to those skilled in the art that numerous variations of the details of the present invention may be made without departing from the invention as defined in the appended claims.

The invention claimed is:

1. A device for evaporative delivery of volatile substances, comprising:
(a) a reservoir portion containing a liquid volatile substance, the reservoir portion having an open cavity with a peripheral portion there around;
(b) a microporous, vapor-permeable membrane having a first surface and a second surface positioned over the reservoir, said membrane being affixed to the peripheral portion of the reservoir and wherein the second surface of the membrane is in contact with at least the liquid volatile substance, the microporous membrane comprising:
(A) a polymeric matrix,
(B) an interconnecting network of pores communicating throughout the polymeric matrix, and
(C) finely divided, substantially water-insoluble filler material,
wherein the microporous membrane further comprises a barrier coating layer over at least the first surface of the microporous membrane; and
(c) a removable cap layer having a first surface and a second surface, wherein an adhesive layer is interposed between the first surface of the microporous membrane and the second surface of the cap layer such that the microporous vapor-permeable membrane and the liquid volatile substance are substantially sealed beneath the cap layer.

2. The device of claim 1, wherein the liquid volatile substance is selected from the group consisting of fragrance release materials, insect repellant release materials, and mixtures thereof.

3. The device of claim 1, wherein the polymeric matrix of the microporous, vapor-permeable membrane (b) comprises at least one thermoplastic polyolefin polymer.

4. The device of claim 1, wherein the pores constitute from 30 to 95 volume percent of the microporous membrane.

5. The device of claim 1, wherein the finely divided, substantially water-insoluble filler material comprises water-insoluble siliceous particles selected from the group consisting of silica, mica, montmorillonite, kaolinite, talc, diatomaceous earth, vermiculite, zeolites, calcium silicate, aluminum silicate, sodium aluminum silicate, aluminum polysilicate, alumina silica gels, and mixtures thereof.

6. The device of claim 5, wherein the siliceous particles are selected from the group consisting of precipitated silica, silica gel, fumed silica, and mixtures thereof.

7. The device of claim 5, wherein the filler material further comprises water-insoluble non-siliceous particles selected from the group consisting of titanium dioxide, zinc oxide, antimony oxide, zirconia, magnesia alumina, zinc sulfide, barium sulfate, strontium sulfate, calcium carbonate, magnesium carbonate, magnesium hydroxide, and mixtures thereof.

8. The device of claim 1, wherein the barrier coating layer comprises a resinous component selected from the group consisting of polyvinyl alcohols, polyvinyl ethers, polyurethanes, polyureas, polyamides, polyvinylidene chlorides, epoxy-amine polymers, poly(meth)acrylates, polyesters, polysiloxanes, and mixtures thereof.

9. The device of claim 8, wherein the resinous component of the barrier coating layer comprises polyvinyl alcohols.

10. The device of claim 1, wherein the barrier coating layer is present on at least one of the first surface and the second surface of the microporous membrane at a coating weight ranging from 0.75 to 5.0 grams per square meter.

11. The device of claim 10, wherein the barrier coating layer is present on at least one of the first surface and the second surface of the microporous membrane at a coating weight ranging from 1.0 to 3.0 grams per square meter.

12. The device of claim 8, wherein the barrier coating layer further comprises a high-aspect ratio pigment selected from the group consisting of vermiculite, mica, talc, metal flakes, platy clays, and platy silicas.

13. The device of claim 1, wherein the removable cap layer is a peel seal.

14. The device of claim 1, wherein the removable cap layer comprises at least one layer selected from the group consisting of metal foils, polymeric films, and combinations thereof.

15. The device of claim 1, wherein the removable cap layer comprises a polymeric film which has been printed to appear metallized.

16. The device of claim 1, wherein the adhesive layer is in contact with the peripheral portion of the reservoir and is in contact with and extends over the at least one barrier coating layer on the first surface of the microporous vapor-permeable membrane.
